(19) United States
(12) Patent Application Publication
(10) Pub. No.: US 2018/0000366 A1
(43) Pub. Date: Jan. 4, 2018

(54) BIOLOGICAL INFORMATION DETECTION APPARATUS

(71) Applicant: SEIKO EPSON CORPORATION, Tokyo (JP)

(72) Inventors: Yoshihiro HIDAI, Shiojiri-shi (JP); Noriaki HIRAIDE, Chino-shi (JP); Hirofumi YANAGISAWA, Shiojiri-shi (JP)

(73) Assignee: SEIKO EPSON CORPORATION, Tokyo (JP)

(21) Appl. No.: 15/635,903

(22) Filed: Jun. 28, 2017

(30) Foreign Application Priority Data
Jul. 1, 2016 (JP) 2016-131383

(51) Int. Cl. A61B 5/024; A61B 5/00

(52) U.S. Cl. CPC A61B 5/02438; A61B 5/681

(57) ABSTRACT

A biological information detection apparatus includes a case, a detection part that houses a light receiving portion that receives light from a test subject and protrudes outer than a case outer surface of the case on the test subject side, and a first convex portion and a second convex portion provided with the detection part in between in a plan view of the test subject side of the case. There is at least a region in which the first convex portion and the second convex portion are not joined from the detection part side over the outer edge side of the case in the plan view.

### CROSS REFERENCE TO RELATED APPLICATION

The current; application claims priority to Japanese patent application 2016-131383, filed Jul. 1, 2016, the entirety of which is incorporated herein by reference.

### BACKGROUND

### 1. Technical Field

The present invention relates to a biological information detection apparatus.

### 2. Related Art

In related art, as biological information detection apparatuses, e.g. a measurement module worn on a part such as a wrist (test subject) using a band or the like and measuring biological information of pulse wave or the like of a wearer and a wrist apparatus in a wristwatch shape having a measurement function of the biological information are known. For example, Patent Document 1 (Publication of US Patent Application No. 2014/0107493) discloses a wrist apparatus worn on an arm (wrist) of a wearer (user) as a test subject and measuring biological information including pulse wave and pulse rate using an optical detection sensor.

In the wrist apparatus (biological information detection apparatus) using the optical detection sensor, biological information including pulse wave and pulse rate is obtained by optical measurement and conversion of the blood flow in the skin surface as a measuring object into signals, It is important to reduce the noise component contained in the light received by the optical detection sensor for improving measurement accuracy.

However, the wrist apparatus described in Patent. Document 1 has a configuration in which the optical detection sensor is protruded from the case so that the optical detection sensor may be in close contact with the arm of the wearer as the user. When the optical detection sensor protrudes from the case, the optical detection sensor is in close contact with the surface of the arm, however, in a region other than the optical detection sensor, a gap is produced between the case surface on the optical detection sensor side and the surface of the arm of the wearer (user), and the wrist apparatus easily swings and tilts due to the gap. When the wrist apparatus swings and tilts, the close contact between the arm of the wearer and the optical detection sensor is no longer maintained, and thereby, the noise component contained in the light increases due to disturbance light and the detection of the biological information including pulse wave and pulse rate may be unstable.

### SUMMARY

An advantage of some aspects of the invention is to solve at least a part of the problems described above, and the invention can be implemented as the following forms or application examples.

### APPLICATION EXAMPLE 1

A biological information detection apparatus according to this application example includes a case, a detection part that houses a light receiving portion that receives light from a test subject and protrudes outer than a case outer surface of the case on the test subject side, and a convex part with a cutout provided in at least a part in a plan view of the test subject side of the case.

Further, the convex part includes a first convex portion and a second convex portion, and the cutout is a region in which the first convex portion and the second convex portion are not joined in the plan view.

According to this application example, when the case (biological information detection apparatus) is attached to an attachment part of the user, in addition to the detection part, the first convex portion and the second convex portion come into contact with the attachment part of the user, and the tilt of the case about the detection part as a fulcrum may be reduced. Thereby, the attachment (fixation) attitude of the biological information detection apparatus becomes stable, and close contact between the attachment part of the user and the detection part may be maintained and the detection of biological information including e.g. pulse wave and pulse rate may be stabilized.

### APPLICATION EXAMPLE 2

In the biological information detection apparatus according to the application example, it is preferable that the first convex portion and the second convex portion are provided closer to an outer edge side of the case than the detection part side.

According to this application example, the first convex portion and the second convex portion are provided closer to the outer edge side of the case and, when the biological information detection apparatus is attached to the attachment part of the user, the case is harder to be tilted and easier to be stable.

### APPLICATION EXAMPLE 3

In the biological information detection apparatus according to the application example, it is preferable that a surface of the region is the same surface as the case outer surface.

According to this application example, the first convex portion and the second convex portion can be provided by effectively utilizing the case outer surface.

### APPLICATION EXAMPLE 4

In the biological information detection apparatus according to the application example, it is preferable that a pair of band parts are provided with the case in between in the plan view, and the first convex portion and the second convex portion are provided along a direction crossing direction in which the pair of band parts are arranged in the plan view.

According to this application example, with respect to the direction in which the pair of band parts are arranged, the motion including the tilt of the case may be reduced in the direction crossing the direction in which the pair of band parts are arranged as the direction in which the motion of the case is relatively easily made.

### APPLICATION EXAMPLE 5

In the biological information detection apparatus according to the application example, it is preferable that external connecting terminals are provided in a region between the detection part and the first convex portion and a region between the detection part and the second convex portion.

According to this application example, the external connecting terminals are provided in the region surrounded by the first convex portion and the second convex portion, and direct contact with the external connecting terminals from outside is harder to occur and damage on the external connecting terminals due to the contact may be reduced.

### APPLICATION EXAMPLE 6

In the biological information detection apparatus according to the application example, it is preferable that external connecting terminals are located in a region opposite to the detection part side with respect to the first convex portion and the second convex portion in the plan view.

According to this application example, the external connecting terminals are located on the outer side than the first convex portion and the second convex portion, and connection to the external connecting terminals is easily made.

### APPLICATION EXAMPLE 7

In the biological information detection apparatus according to the application example, it is preferable that external connecting terminals are located on a surface of the region.

According to this application example, the external connecting terminals are provided on the surface of the region in which the first convex portion and the second convex portion are located on both sides, and thereby, direct contact with the external connecting terminals from outside is harder to occur and damage on the external connecting terminals due to the contact may be reduced.

### APPLICATION EXAMPLE 8

In the biological information detection apparatus according to the application example, it is preferable that external connecting terminals are located in at least one of the first convex portion and the second convex portion.

According to this application example, damage on the external connecting terminals form outside may be reduced and the connection is easily made.

### APPLICATION EXAMPLE 9

In the biological information detection apparatus according to the application example, it is preferable that the first convex portion and the second convex portion are detachable from the case.

According to this application example, the first convex portion and the second convex portion may be used only when the first convex portion and the second convex portion are necessary, and designability may be improved.

### APPLICATION EXAMPLE 10

In the biological information detection apparatus according to the application example, it is preferable that the first convex portion and the second convex portion are provided movably with respect to the case.

According to this application example, the first convex portion and the second convex portion may be moved as appropriate and placed in gaps between one surface of the case and the attachment part. In this manner, the first convex portion and the second convex portion may be easily attached.

### APPLICATION EXAMPLE 11

In the biological information detection apparatus according to the application example, it is preferable that the first convex portion and the second convex portion are connected to be rotationally moved with respect to the case.

According to this application example, the first convex portion and the second convex portion may be rotated and easily placed in the positions outside of the detection part. Note that the above described rotation movement is to rotate the first convex portion and the second convex portion and move the positions thereof about the rotation shafts fixed to the case.

### APPLICATION EXAMPLE 12

In the biological information detection apparatus according to the application example, it is preferable that the case includes storage portions that house the first convex portion and the second convex portion.

According to this application example, the first convex portion and the second convex portion are housed in the case (storage portions), and thereby, projection of the first convex portion and the second convex portion when not necessary may be prevented and the designability may be improved.

### APPLICATION EXAMPLE 13

In the biological information detection apparatus according to the application example, it is preferable that sections of parts of the first convex portion and the second convex portion in contact with the test subject have curved shapes.

According to this application example, the sections of the parts of the first convex portion and the second convex portion in contact with the test subject have curved shapes, and thereby, the detection part may be pressed against the test subject in a stable condition with reduced uncomfortable feeling of the user.

### APPLICATION EXAMPLE 14

In the biological information detection apparatus according to the application example, it preferable that the first convex portion and the second convex portion are circumferentially provided to surround the detection part in the plan view.

According to this application example, the designability relating to the arrangement of the first convex portion and the second convex portion may be improved. Further the tilt of the case attached to the attachment part of the user may be effectively reduced.

### BRIEF DESCRIPTION OF THE DRAWINGS

The invention will be described with reference to the accompanying drawings, wherein like numbers reference like elements.

FIG. 1A is a top (front surface) perspective view showing a configuration example of a biological information detection apparatus according to the first embodiment.

FIG. 1B is a bottom (back surface) perspective view showing the biological information detection apparatus according to the first embodiment.

FIG. 2A is a side view showing a configuration example of a biological information detection apparatus of related art.

FIG. 2B is an explanatory diagram of a problem of the biological information detection apparatus of related art.

FIG. 3 shows the configuration example of a biological information detection apparatus according to the first embodiment in a plan view of a case as seen from the back surface side.

FIG. 4A is a side view of the case as seen from an extension direction (connected direction) of band parts.

FIG. 4B is a sectional view of FIG. 4A of the case as seen from the extension direction (connected direction) of the band parts.

FIG. 4C is a sectional view showing a curved shape of a first convex portion (second convex portion) in contact with a test subject.

FIG. 5 shows another arrangement example 1 of the first convex portion (second convex portion) in a plan view of the case as seen from the back surface side.

FIG. 6 shows another arrangement example 2 of the first convex portion (second convex portion) in the plan view of the case as seen from the back surface side.

FIG. 7 shows another arrangement example 3 of the first convex portion (second convex portion) in the plan view of the case as seen from the back surface side.

FIG. 8 shows another arrangement example 4 of the first convex portion (second convex portion) in the plan view of the case as seen from the back surface side.

FIG. 9 shows another arrangement example 1 of external connecting terminals in the plan view of the case as seen from the back surface side.

FIG. 10 shows another arrangement example 2 of the external connecting terminals in the plan view of the case as seen from the back surface side.

FIG. 11 shows another arrangement example 3 of the external connecting terminals in the plan view of the case as seen from the back surface side.

FIG. 12 is a side view showing another configuration example of the first convex portion (second convex portion) as seen from the same direction as that in FIG. 4A.

FIG. 13A shows a configuration example of biological information detection apparatus according to the second embodiment in a plan view of a case as seen from the back surface side.

FIG. 13B is a side view of the case as seen from an extension direction (connected direction) of band parts.

FIG. 14 shows a configuration example of a biological information detection apparatus according to the third embodiment in a side view of a case as seen from an extension direction (connected direction) of band parts.

FIG. 15A shows a configuration example of a biological information detection apparatus according to the fourth embodiment in a side view of a case as seen from an extension direction (connected direction) of band parts.

FIG. 15B is a perspective view showing a first convex portion second convex portion) in the fourth embodiment.

FIG. 16 is a bottom (back surface) perspective view showing a biological information detection apparatus according to a modified example.

### DESCRIPTION OF EXEMPLARY EMBODIMENTS

As below, embodiments will be explained. Note that the embodiments to be explained do not unduly limit the invention described in the appended claims. Further, not all of the configurations to be explained in the embodiments are necessarily essential component elements of the invention.

### First Embodiment

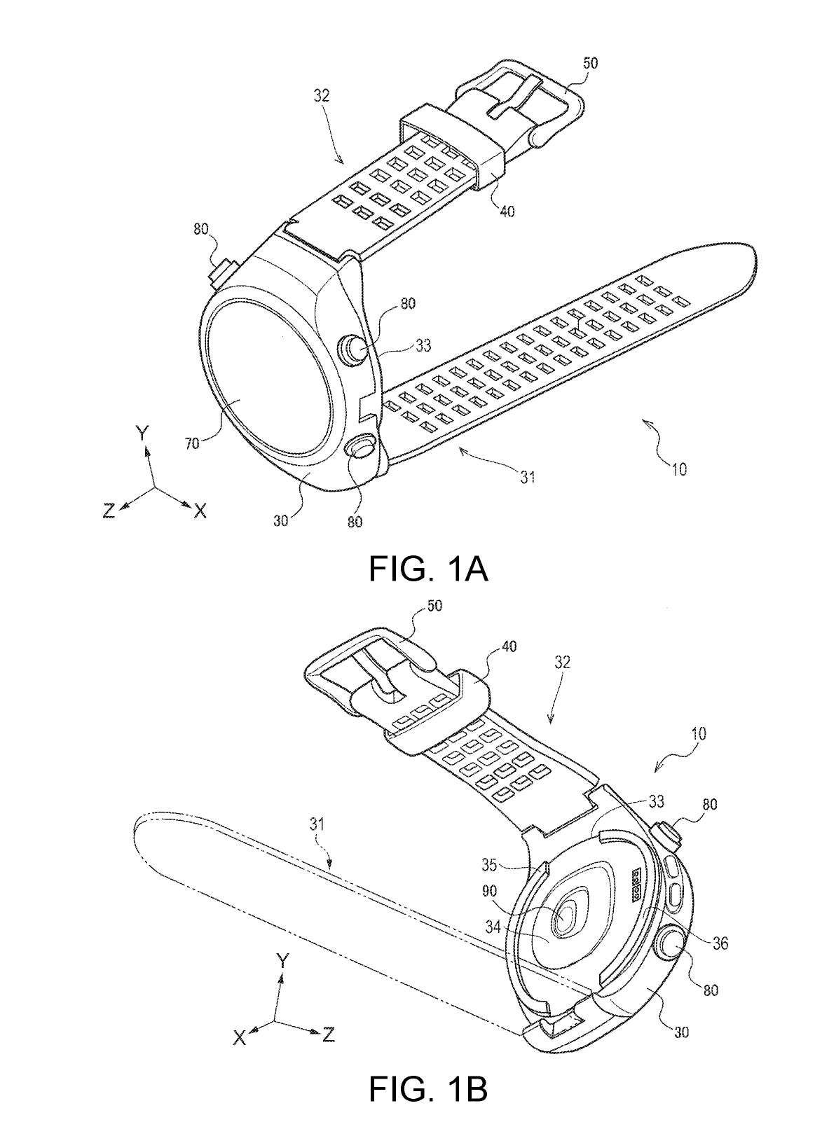

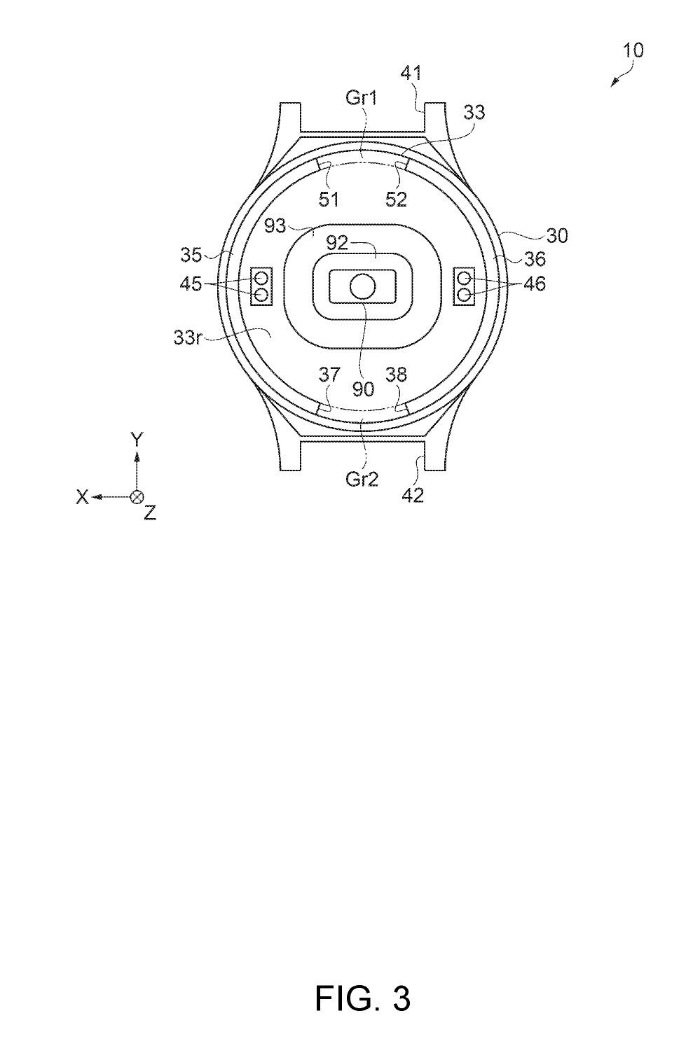

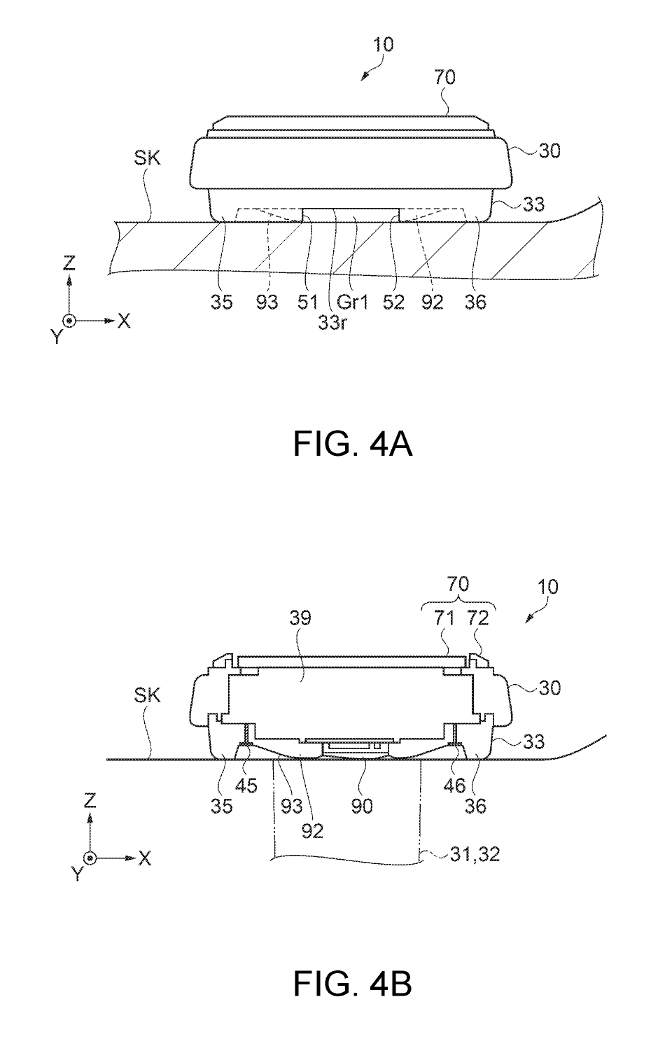

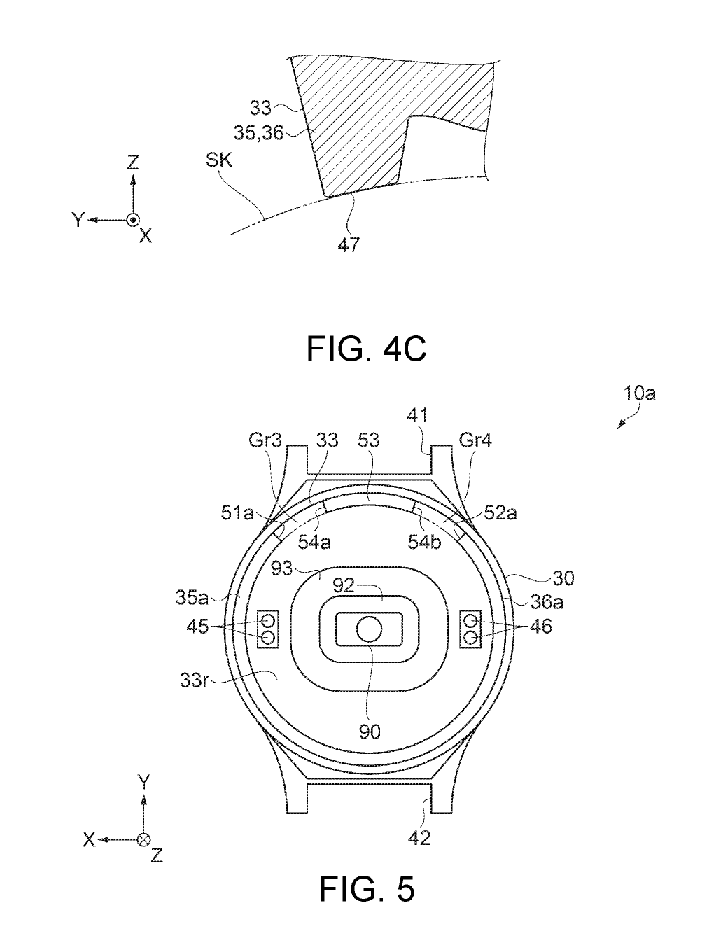

A configuration example of a biological information detection apparatus according to the first embodiment of the invention will be explained using FIGS. 1A, 1B, 3, 4A, 4B, and 4C. FIG. 1A is a perspective view of the biological information detection apparatus according to the invention from the top (front surface) side, and FIG. 1B is a perspective view of the biological information detection apparatus according to the first embodiment of the invention from the bottom (front surface) side. FIGS. 3, 4A, 4B, and 4C show the configuration example of the biological information detection apparatus according to the first embodiment. FIG. 3 is a plan view of a case as seen from the back surface side, FIG. 4A is a side view of FIG. 4A of the case as seen from an extension direction (connected direction) of band parts, FIG. 4B is a sectional view of FIG. 4A of the case as seen from the extension direction (connected direction) of the band parts, and FIG. 4C is a sectional view showing a curved shape of a part of a first convex portion (second convex portion) in contact with a test subject Note that, in the specification, the explanation will be made with the display surface side of the biological information detection apparatus as the top side or front side and the opposite side to the display surface (the side in contact with the test subject when worn) as the bottom side or back side. Further, in FIGS. 1A, 1B, 3, 4A, 4B, and 4C, for convenience of explanation, the explanation will be made using three axes orthogonal to one another as an X-axis, Y-axis, Z-axis, and an axis perpendicular to the surface of a glass plate 71, which will be described later, is the Z-axis and the direction in which a pair of band parts (first band part 31 and second band part 32) placed with the case in between are arranged is the Y-axis. Furthermore, as below, “axis direction” refers to “direction along axis”.

As shown in FIGS. 1A and 1B, in the biological information detection apparatus 10 according to the first embodiment, a bottom case (lid part) 33 as a case attached to a test subject SK as shown in FIG. 4A in which a sensor part (sensor) 90 containing a light sensor that detects biological information of the test subject SK is provided and a top case 30 located on the opposite side to the attachment side to a user are provided. Further, band parts (first band part 31 and second band part 32) are respectively connected to both sides of a case part including the bottom case 33 and the top case 30 along the Y-direction.

The configuration of the biological information detection apparatus 10 is explained in further detail. The biological information detection apparatus 10 includes the first band part 31, the second band part 32, the case part containing the bottom case (lid part) 33 and the top case 30, a fixing part (free loop, fixed loop) 40, a connecting part (buckle) 50, a display part 70, an operation part 80, and the sensor part 90 as a light sensor part. Note that the biological information detection apparatus 10 is not limited to the configuration in FIGS. 1A and 1B, but various modifications such that part of these component elements are omitted and other component elements are added can be made.

The bottom case 33 and the top case 30 forming the case part may be formed using e.g. a metal such as stainless steel or resin. Note that the case part is not limited to that having the form separated into the top case 30 and the bottom case 33, but a configuration in which a case having an integrated structure (corresponding to the top case 30) and a back lid (corresponding to the bottom case 33) provided on the attachment side of the case to the user may be employed.

As shown in FIGS. 4A and 4B, a bezel 72 is provided on the top side (top case 30) opposite to the side of the biological information detection apparatus 10 attached to the test subject SK, and the glass plate 71 as a top plate part (outer wall) placed inside of the bezel 72 and protecting the internal structure is provided. The biological information detection apparatus 10 includes the display part 70 (display) containing a dial plate (not shown) provided immediately below the glass plate 71 via the glass plate 71. The user may view the display of the display part 70. That is, in the biological information detection apparatus 10 of the embodiment, the detected biological information may be displayed on the display part 70 and the display may be presented from the top side of the biological information detection apparatus 10 to the user.

Note that, here, the example in which the top plate part of the biological information detection apparatus 10 is realized by the glass plate 71 is shown, however, the top plate part can be formed using another material such as transparent plastic than glass by a transparent member through which the display part 70 can be viewed with strength that can protect the configurations contained inside (internal space 39) of the top case 30 and the bottom case 33 including the display part 70. The configuration example with the bezel 72 is shown, however, a configuration without the bezel 72 may be employed.

On the case outer surface of the bottom case (lid part) 33 located on the test subject SK side, a detection part 92 (detector) housing the sensor part 90 containing the light sensor that detects biological information of the test subject SK and a back surface 33r placed on the outer side than the outer edge of the detection part 92 (the side on which the outer edge of the bottom case 33 is located) are provided.

Further, as shown in FIGS. 1A and 1B, on the side surface of the biological information detection apparatus 10, the operation part 80 including a plurality of buttons that enables e.g. switching of detection modes by the sensor part 90 and switching of display modes displayed on the display part 70 is provided. The display part 70 displays various kinds of information. The operation part 80 receives operation by the user.

In the internal space 39 formed by the bottom case 33, the top case 30, the glass plate 71, etc. shown in FIGS. 4A and 4B, the display part 70 including the dial plate (not shown), a control part (not shown) (controller) that processes data detected by the sensor part 90 and controls display contents of the display part 70, a battery (not shown), etc. are housed. Note that, in FIGS. 4A and 4B, the configurations within the internal space 39 are not shown.

The first band part 31 is attached to one end of the top case 30 (a first band attachment part 41 shown in FIG. 3) and the second band part 32 is attached to the other end of the top case 30 (a second band attachment part 42 shown in FIG. 3).

Then, the connecting part (buckle) 50 connects the first band part 31 and the second band part 32. The fixing part (free loop, fixed loop) 40 fixes the first band part 31 along the second band part 32 with the first band part 31 and the second band part 32 connected.

The sensor part 90 is housed in the detection part 92 provided to protrude in a convex shape from the back surface 33r as the case outer surface of the bottom case (lid part) 33 located on the test subject SK side. In other words, the sensor part 90 further protrudes toward the test subject SK side (Z-axis direction) than the back surface 33r. As shown in FIGS. 3 and 4B, the detection part 92 is an apex part extended. from the back surface 33r via a slope 93, and the sensor part 90 including the light sensor is housed in the center portion. The sensor part 90 is pressed against the test subject SK with the detection part 92 to detect biological information of the test subject SK.

In the sensor part 90 as the light sensor part, a light emitting portion and a light receiving portion are provided (not shown). The light emitting portion outputs light to the test subject SK. The light receiving portion receives the light (reflected light, transmitted light) from the test subject SK. For example, when the light emitting portion outputs light to the test subject SK and the light is reflected by the test subject SK (blood vessel), the light receiving portion receives the reflected light and may detect the biological information. The light receiving portion may be realized by e.g. a light receiving element such as a photodiode and the light emitting portion may be realized by a light emitting element e.g. an LED.

A pulsimeter is taken as an example. The light from the light emitting portion travels within the test subject SK, and is diffused or scattered by epidermis, dermis, subcutaneous tissue etc. Then, the light reaches the blood vessel (part to be detected) and is reflected. In this regard, part of the light is absorbed by the blood vessel. Then, absorption of the light in the blood vessel changes due to the pulse rate and the light quantity of the reflected light also changes, and thereby, the pulse rate etc. as biological information may be detected by the light receiving portion receiving the reflected light and detecting the change of the light quantity.

Further, as shown in FIGS. 3, 4A and 4B, in the plan view of the back surface 33r of the bottom case 33, namely, when the back surface 33r of the bottom case 33 is seen from the Z-axis direction, a first convex portion 35 and a second convex portion 36 placed apart from the detection part 92 in the X-axis direction in positions with the part in between or surrounding the part are provided in a pair in the back surface 33r. In other words, the first convex portion 35 and the second convex portion 36 are provided in the positions with the detection part 92 in between in a direction crossing the direction (Y-axis direction) in which the first band part 31 and the second band part 32 are arranged, in the embodiment, along the X-axis direction. Further, the first convex portion 35 and the second convex portion 36 as part of a circumferential shape surrounding the detection part 92 protrude from the back surface 33r of the bottom case 33 in the same direction as the protrusion direction of the detection part 92 (Z-axis direction). The heights of the first convex portion 35 and the second convex portion 36 from the back surface 33r may be generally the same as the height of the detection part 92 from the back surface 33r, and more preferably the same. Further, the first convex portion 35 and the second convex portion 36 are extended in an arc shape along at least a part of the outer edge of the bottom case 33.

That is, the biological information detection apparatus 10 includes the case, the detection part housing the light receiving portion that receives light from the test subject and protruded from the case outer surface of the case on the test subject side, and a convex part arranged side by side with the detection part in the plan view from the test subject side of the case and provided with a cutout in at least a part.

Further, the convex part includes the first convex portion and the second convex portion and the cutout is a region in which the first convex portion and the second convex portion are not joined in the plan view.

Furthermore, in the plan view, the convex part is circumferentially provided to surround the detection part.

The first convex portion 35 has one end 51 on one side in the extension direction and has another end 37 on the other opposite side. Further, the second convex portion 36 has one end 52 on one side in the extension direction and has another end 38 on the other opposite side. In the extension direction of the first convex portion 35 and the second convex portion 36, at least one groove portion (in the embodiment, two of a first groove portion Gr1 and a second groove portion Gr2) cut out from the detection part 92 side over the outer edge of the bottom case 33 (the region in which the first convex portion 35 and the second convex portion 36 are not joined) is provided.

Specifically, the first groove portion Gr1 cut out from the detection part 92 side over the outer edge side of the bottom case 33 is provided between the one end 51 of the first convex portion 35 and the one end 52 of the second convex portion 36 located in their extension directions. In other words, the first convex portion 35 and the second convex portion 36 are not joined to each other from the detection part 92 side over the outer edge side of the bottom case 33 by the first. groove portion Gr1. Further, the second groove portion Gr2 cut out from the detection part 92 side over the outer edge side of the bottom case 33 is provided between the other end 37 of the first convex portion 35 and the other end 38 of the second convex portion 36 located in their extension directions. In other words, the first convex portion 35 and the second convex portion 36 are not joined to each other from the detection part 92 side over the outer edge side of the bottom case 33 by the second groove portion Gr2. Note that, in the embodiment, the first groove portion Gr1 and the second groove portion Gr2 are provided in the positions on the opposite sides along the Y-axis direction, in other words, in the positions with the detection part 92 in between in the Y-axis direction.

As described above, the first convex portion 35 and the second convex portion 36 are provided at least in a pair divided by the at least one groove portion (in the embodiment, two of the first groove portion Gr1 and the second groove portion Gr2) cut out from the detection part 92 side over the outer edge side of the bottom case 33. Thereby, when the biological information detection apparatus 10 is attached to the attachment part (test subject SK) of the user, the tilt of the case part (bottom case 33 and the top case 30) with respect to the directions on both sides with the detection part 92 in between may be reduced.

It is preferable that the first convex portion 35 and the second convex portion 36 are provided as a part of the circumferential shape surrounding the detection part 92 in the plan view from the Z-axis direction. By the arrangement of the first convex portion 35 and the second convex portion 36, designability according to the arrangement of the first convex portion 35 and the second convex portion 36 may be improved. Further, the occurrence of the tilt of the biological information detection apparatus 10 attached to the attachment part (test subject SK) of the user or the like may be effectively reduced.

Note that the first groove portion Gr1 and the second groove portion Gr2 are cut out to the back surface 33r (case outer surface) of the bottom case 33. That is, the respective bottom surfaces of the first groove portion Gr1 and the second groove portion Gr2 are the same as the back surface 33r (case outer surface) of the bottom case 33 and the surface extended from the back surface 33r is exposed within the first groove portion Gr1 and the second groove portion Gr2. In this manner, the first convex portion 35 and the second convex portion 36 can be provided by effectively utilizing the case outer surface (the back surface 33r of the bottom case 33).

Further, as shown in FIG. 4C, it is preferable that, in the first convex portion 35 and the second convex portion 36, sections of contact surfaces 47 as parts in contact with the test subject SK are formed in curved shapes conforming the outer surface shape of the test subject SK such as an arm of the user, for example, to which the biological information detection apparatus 10 is attached. As described above, the contact surfaces 47 with the test subject SK are formed in the curved shapes, and thereby, the detection part 92 may be pressed against the test subject SK in a stable condition with reduced uncomfortable feeling of the user.

Furthermore, the first convex portion 35 and the second convex portion 36 may be integrally provided with the bottom case 33 or separately molded from the bottom case 33 and attached to the back surface 33r of the bottom case 33 by bonding.

Moreover, it is preferable that the first convex portion 35 and the second convex portion 36 are formed using a soft material including e.g. urethane resin, silicone resin, elastomer resin, fluorine resin, polystyrene resin, and polyethylene resin. Or, it is preferable to provide the above described soft material in the tip end portions including the contact surfaces 47 of the first convex portion 35 and the second convex portion 36 with the test subject SK. As described above, the soft material is used for the first convex portion 35 and the second convex portion 36, and thereby, the uncomfortable feeling of the user may be reduced.

As shown in FIG. 3, in the plan view of the back surface 33r of the bottom case 33, namely, when the back surface 33r of the bottom case 33 is seen from the Z-axis direction, a pair of external connecting terminals (first external connecting terminals 45 and second external connecting terminals 46) are provided in a region between the detection part 92 and the first convex portion 35 and a region between the detection part 92 and the second convex portion 36 in the X-axis direction. The first external connecting terminals 45 and the second external connecting terminals 46 are connecting terminals for connecting terminals for signal transmission between an external apparatus and themselves and power supply. As described above, the first external connecting terminals 45 and the second external connecting terminals 46 are provided in the regions having outer circumferences surrounded by the first convex portion 35 and the second convex portion 36, and thereby, occurrence of damage on the first external connecting terminals 45 and the second external connecting terminals 46 from outside may be reduced.

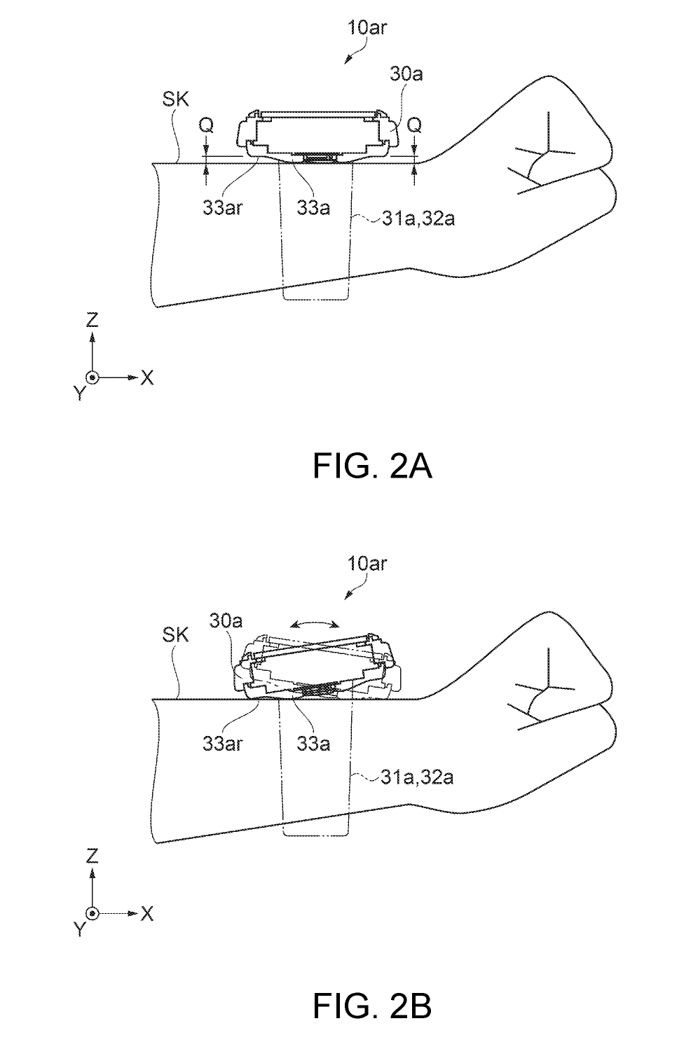

On the other hand, FIGS. 2A and 2B exemplify a biological information detection apparatus 10ar of related art described as above. Note that FIG. 2A is a side view showing a configuration example of the biological information detection apparatus of related art, and FIG. 2B is an explanatory diagram for explanation of a problem of the biological information detection apparatus of related art. The biological information detection apparatus 10ar shown in FIGS. 2A and 2B is different from the biological information detection apparatus 10 shown in FIGS. 1A and 1B in that the first convex portion 35 and the second convex portion 36 of the back surface 33r of the bottom case 33 are not provided.

In the biological information detection apparatus 10ar of related art without the first convex portion 35 and the second convex portion 36 of the back surface 33r of the bottom case 33, gaps Q are produced between a back surface 33ar of a bottom case 33a and the arm surface (test subject SK). In the biological information detection apparatus 10ar as shown in FIGS. 2A and 2B, a large acceleration of gravity may be applied to the apparatus main body formed by a top case 30a and the bottom case 33a due to severe swing of the arm or the like. When the acceleration of gravity is applied in the Z-axis direction, the apparatus main body formed by the top case 30a, bottom case 33a, etc. may swing, twist, or tilt according to the motion of the arm as shown by an arrow in FIG. 2B due to the presence of the gaps Q between the back surface 33ar of the bottom case 33a and the arm surface (test subject SK). Note that the phenomenon of swing, twist, or tilt notably occurs in the direction in which the main body is not fixed by band parts 31a, 32a (X-axis direction).

As described above, when the apparatus main body formed by the top case 30a, bottom case 33a, etc. swings, twists, or tilts, the detection part 92 and the sensor part 90 may partially float from the test subject SK. Accordingly, normal acquisition of the sensor signals by the sensor part 90 becomes impossible and a problem of unstable detection of biological information including e.g. pulse wave and pulse rate arises.

Accordingly, in the embodiment, as shown in FIGS. 1A, 1B, 3, 4A, and 4B, in the plan view of the back surface 33r of the bottom case 33 from the Z-axis direction, the first convex portion 35 and the second convex portion 36 are provided in the pair in the positions with the detection part 92 in between in the X-axis direction. Thereby, when the apparatus main body formed by the top case 30a, bottom case 33a, etc. (biological information detection apparatus 10) is attached to the attachment part (test subject SK) of the user, in addition to the detection part 92, the first convex portion 35 and the second convex portion 36 come into contact with the test subject. SK, and the tilt of the case part about the detection part 92 as a fulcrum may be suppressed. In other words, even when the wearer does heavy exercise and the test subject SK moves, the state in which the sensor part 90 (detection part 92) is pressed against the test subject SK with given pressure can be maintained. As described above, the attachment (fixation) attitude of the biological information detection apparatus 10 is stable, and thereby, false detection of the sensor signal according to the biological information including e.g. pulse wave and pulse rate may be prevented and the detection of the biological information may be stabilized.

Further, the first convex portion 35 and the second convex portion 36 are provided in the positions with the detection part 92 in between along the direction crossing the direction in which the first band part 31 and the second band part 32 are connected (Y-axis direction), in the embodiment, along the X-axis direction. Regarding the biological information detection apparatus 10, the bottom case 33 and the top case 30 are attached to the test subject by the first band part 31 and the second band part 32, and swing and tilt are hard to occur and the attitude is easily stabilized in the connecting direction (Y-axis direction), however, swing and tilt are relatively easy to occur in the X-axis direction. Therefore, the first convex portion 35 and the second convex portion 36 are provided along the X-axis direction, and thereby, occurrence of swing and tilt of the biological information detection apparatus 10 may be reduced and the detection of the biological information may be stabilized.

Furthermore, by the groove portions (the first groove portion Gr1 and the second groove portion Gr2) provided between the first convex portion 35 and the second convex portion 36, the region in which the detection part 92 is located is not an enclosed space, but communicates with a region outside of the first convex portion 35 and the second convex portion 36, and retention of moisture (water) due to e.g. sweat in the region in which the detection part 92 is located may be reduced. Thereby, unstable detection due to moisture (water) may be reduced and the detection of the biological information by the sensor part 90 may be stabilized.

In addition, the first convex portion 35 and the second convex portion 36 are provided to surround the outer circumference of the sensor part 90 and function as light-blocking walls, and outside light including disturbance light entering the sensor part 90 can be blocked or suppressed.

### Other Arrangement Examples of First Convex Portion and Second Convex Portion

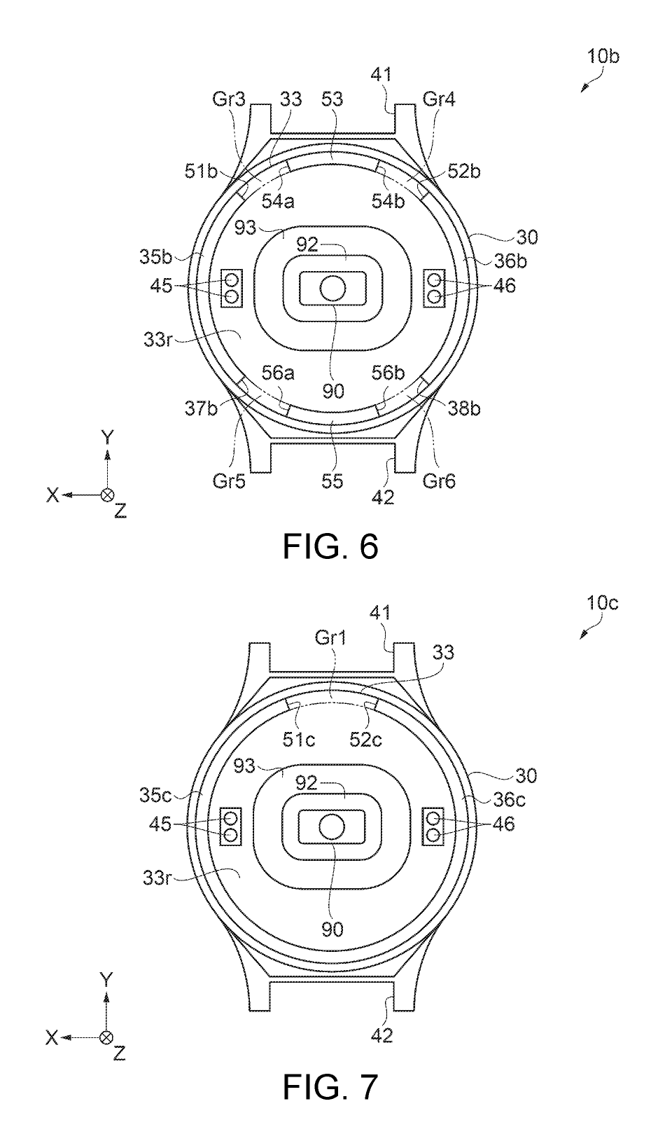

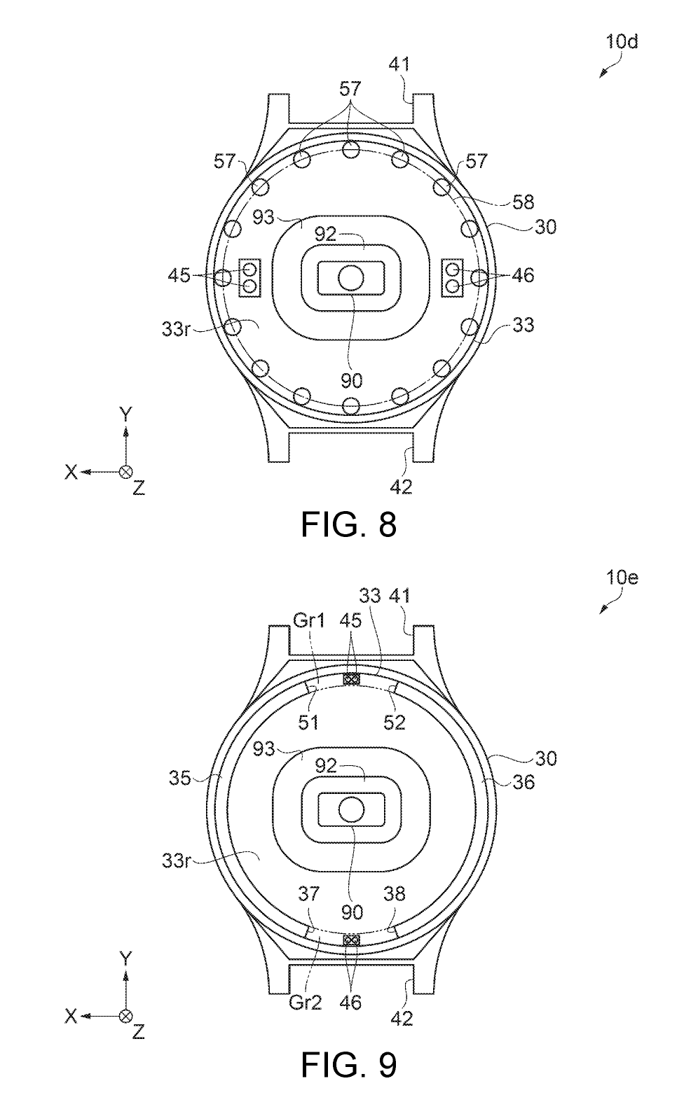

The first convex portion 35 and the second convex portion 36 of the biological information detection apparatus 10 according to the above described first embodiment may be variously arranged. As below, the other arrangement examples of the first convex portion 35 and the second convex portion 36 of the biological information detection apparatus 10 according to the above described first embodiment will be explained using FIGS. 5 to 8. FIGS. 5, 6, 7 and 8 are plan views of bottom cases showing the other arrangement examples of the first convex portion (second convex portion) from the back surface side. FIG. 5 shows the other arrangement example 1 of the first convex portion (second convex portion), FIG. 6 shows the other arrangement example 2 of the first convex portion (second convex portion), FIG. 7 shows the other arrangement example 3 of the first convex portion (second convex portion), and FIG. 8 shows the other arrangement example 4 of the first convex portion (second convex portion). Note that, in the following explanation, the same configuration parts as those of the biological information detection apparatus 10 according to the above described first embodiment will have the same signs and their explanation may be omitted.

First, referring to FIG. 5, the other arrangement example 1 is explained. As shown in FIG. 5, a biological information detection apparatus 10a includes a first convex portion 35a, a second convex portion 36a, and a third convex portion 53 according to the other arrangement example 1. As is the case of the first embodiment, the first convex portion 35a and the second convex portion 36a protrude from the back surface 33r of the bottom case (lid part) 33 to surround the detection part 92 in the same direction as the protrusion direction of the detection part 92 and are provided in the positions with the detection part 92 in between in the X-axis direction.

The first convex portion 35a and the second convex portion 36a have one ends 51a, 52a, respectively, and have an integrated structure in which the opposite sides to the one ends 51a, 52a are joined. The third convex portion 53 is provided between the one end 51a of the first convex portion 35a and the one end 52a of the second convex portion 36a, and has one end 54a provided on the first convex portion 35a side and another end 54b provided on the second convex portion 36a side. Further, a third groove Gr3 cut out from the detection part 92 side over the outer edge of the bottom case 33 is provided between the one end 51a of the first convex portion 35a and the one end 54a of the third convex portion 53. Furthermore, a fourth groove Gr4 cut out from the detection part 92 side over the outer edge of the bottom case 33 is provided between the one end 52a of the second convex portion 36a and the other end 54 of the third convex portion 53. As described above, the first convex portion 35a and the second convex portion 36a are divided by the two groove portions (the third groove Gr3 and the fourth groove Gr4).

Next, referring to FIG. 6, the other arrangement example 2 is explained. As shown in FIG. 6, a biological information detection apparatus 10b includes a first convex portion 35b, a second convex portion 36b, a third convex portion 53, and a fourth convex portion 55 according to the other arrangement example 2. As is the case of the first embodiment, the first convex portion 35b and the second convex portion 36b protrude from the back surface 33r of the bottom case 33 to surround the detection part 92 in the same direction as the protrusion direction of the detection part 92 and are provided in the positions with the detection part 92 in between in the X-axis direction.

The first convex portion 35b has one end 51b and another end 37b on the opposite side. Further, the second convex portion 36b located on the opposite side to the first convex portion 35b via the detection part 92 has one end 52b and another end 38b as an opposite end.

As is the case of the above described other arrangement example 1, the third convex portion 53 is provided between the one end 51b of the first convex portion 35b and the one end. 52b of the second convex portion 36b, and has one end 54a provided on the first convex portion 35b side and another end 54b provided on the second convex portion 36b side. Further, a third groove Gr3 cut out from the detection part 92 side over the outer edge of the bottom case 33 is provided between the one end 51b of the first convex portion 35b and the one end 54a of the third convex portion 53. Furthermore, a fourth groove Gr4 cut out from the detection part 92 side over the outer edge of the bottom case 33 is provided between the one end 52b of the second convex portion 36b and the other end 54b of the third convex portion 53.

The fourth convex portion 55 is located on the opposite side to the third convex portion 53 via the detection part 92. The fourth convex portion 55 is provided between the other end. 37b of the first convex portion 35b and the other end 38b of the second convex portion 36b, and has one end 56a provided on the first convex portion 35b side and another end 56b provided on the second convex portion 36b side. Further, a fifth groove Gr5 cut out from the detection part 92 side over the outer edge of the bottom case 33 is provided between the other end 37b of the first convex portion 35b and the one end 56a of the fourth convex portion 55. Furthermore, a sixth groove Gr6 cut out from the detection part 92 side over the outer edge of the bottom case 33 is provided between the other end 38b of the second convex portion 36b and the other end 56b of the fourth convex portion 55.

Next, referring to FIG. 7, the other arrangement example 3 is explained. As shown in FIG. 7, a biological information detection apparatus 10c includes a first convex portion 35c and a second convex portion 36c. As is the case of the first embodiment, the first convex portion 35c and the second convex portion 36c protrude from the back surface 33r of the bottom case (lid part) 33 to surround the detection part 92 in the same direction as the protrusion direction of the detection part 92 (Z-axis direction) and are provided in the positions with the detection part 92 in between in the X-axis direction. Further, the first convex portion 35c and the second convex portion 36c are extended in an arc shape along at least a part of the outer edge of the bottom case 33.

The first convex portion 35c has an end portion 51c on one side in the extension direction. The second convex portion 36c has an end portion 52c on one side in the extension direction. Further, the first convex portion 35c and the second convex portion 36c have an integrated structure in which the opposite sides to the end portions 51c, 52c are joined. A first groove portion Gr1 cut out from the detection part 92 side over the outer edge side of the bottom case 33 is provided between the end portion 51c of the first convex portion 35c and the end portion 52c of the second convex portion 36c located in their extension directions. In other words, the first convex portion 35c and the second convex portion 36c are divided from the detection part 92 side over the outer edge side of the bottom case 33 by the first groove portion Gr1.

Note that the above described first groove portion Gr1 and second groove portion Gr2 are cut out to the back surface 33r (case outer surface) of the bottom case 33. That is, the respective bottom surfaces of the first groove portion Gr1 and the second groove portion Gr2 are the same as the back surface 33r (case outer surface) of the bottom case 33 and the surface extended from the back surface 33r is exposed within the first groove portion Gr1 and the second groove portion Gr2.

Next, referring to FIG. 8, the other arrangement example 4 is explained. As shown in FIG. 8, a biological information detection apparatus 10d includes a plurality of convex portions 57 having circular outer shapes and planar bottom surfaces provided on a circumference of a circle (virtual circle 58) surrounding the detection part 92 or a circumference of an ellipse via respective regions (groove portions) as gaps. As is the case of the first embodiment, the convex portions 57 protrude from the back surface 33r of the bottom case (lid part) 33 to surround the detection part 92 in the same direction as the protrusion direction of the detection part 92.

Note that the virtual circle 58 is temporarily provided so that external connecting terminals (first external connecting terminals 45 and second external connecting terminals 46) are provided in a region between the convex portions 57 and the detection part 92. Further, the surface of the respective regions (groove portions) as the gaps between the convex portions 57 may be the same surface as the back surface 33r of the bottom case 33.

### Other Arrangement Examples of External Connecting Terminals

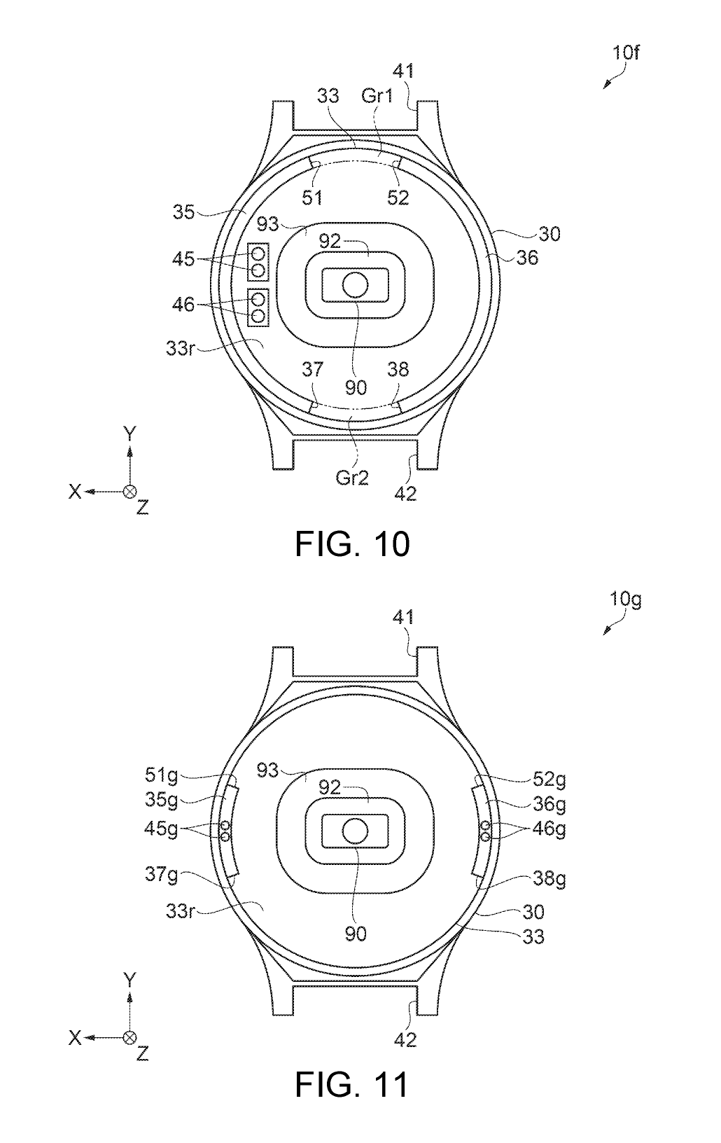

The external connecting terminals (first external connecting terminals 45 and second external connecting terminals 46) of the biological information detection apparatus 10 according to the above described first embodiment may be variously arranged. As below, the other arrangement examples of the external connecting terminals (first external connecting terminals 45 and second external connecting terminals 46) of the biological information detection apparatus 10 according to the above described first embodiment will be explained using FIGS. 9, 10, and 11. FIGS. 9, 10, and 11 are plan views of bottom cases showing the other arrangement examples of the external connecting terminals from the back surface side. FIG. 9 shows the other arrangement example 1 of the external connecting terminals, FIG. 10 shows the other arrangement example 2 of the external connecting terminals, and FIG. 11 shows the other arrangement example 3 of the external connecting terminals. Note that, in the following explanation, the same configuration parts as those of the biological information detection apparatus 10 according to the above described first embodiment will have the same signs and their explanation may be omitted.

First, referring to FIG. 9, the other arrangement example 1 of the external connecting terminals is explained. As shown in FIG. 9, a biological information detection apparatus 10e includes a first convex portion 35 and a second convex portion 36 like those of the first embodiment. When the back surface 33r of the bottom case (lid part) 33 is seen from the Z-axis direction, the first convex portion 35 and the second convex portion 36 are extended along the circumference of the detection part 92, protrude from the back surface 33r of the bottom case 33 in the same direction as the protrusion direction of the detection part 92, and are provided in the positions with the detection part 92 in between in the X-axis direction.

The first external connecting terminals 45 are provided in the region of a first groove Gr1 located between one end 51 of the first convex portion 35 and one end 52 of the second convex portion 36. Further, the second external connecting terminal 46 are provided in the region of a second groove Gr2 located between the other end 37 of the first convex portion 35 and the other end 38 of the second convex portion 36.

As described above, the first external connecting terminals 45 and the second external connecting terminals 46 are provided on the bottom surface (back surface 33r) of the region of the first groove Gr1 or the region of the second groove Gr2 on both sides of which the first convex portion 35 and the second convex portion 36 are located, and thereby, contact with the external connecting terminals (first external connecting terminals 45 and second external connecting terminals 46) from outside harder to occur. Therefore, damage on the first external connecting terminals 45 and the second external connecting terminals 46 from outside may be suppressed. Further, the first external connecting terminals 45 and the second external connecting terminals 46 may be efficiently arranged and space efficiency may be improved.

Next, referring to FIG. 10, the other arrangement example 2 of the external connecting terminals is explained. As shown in FIG. 10, a biological information detection apparatus 10f includes a first convex portion 35 and a second convex portion 36 like those of the first embodiment. When the back surface 33r of the bottom case (lid part) 33 is seen from the Z-axis direction, the first convex portion 35 and the second convex portion 36 are extended along the circumference of the detection part 92, protrude from the back surface 33r of the bottom case 33 in the same direction as the protrusion direction of the detection part 92, and are provided in the positions with the detection part 92 in between in the X-axis direction.

Further, when the back surface 33r of the bottom case (lid part) 33 is seen from the Z-axis direction, external connecting terminals (first external connecting terminals 45 and second external connecting terminals 46) are provided side by side along the Y-axis direction in a region between the detection part 92 and the first convex portion 35 in the X-axis direction. The first external connecting terminals 45 and the second external connecting terminals 46 are connecting terminals for connecting terminals for signal transmission between an external apparatus and themselves and power supply.

As described above, the first external connecting terminals 45 and the second external connecting terminals 46 are provided in the region between the first convex portion 35 and the detection part 92, and thereby, damage on the first external connecting terminals 45 and the second external connecting terminals 46 from outside may be suppressed.

Next, referring to FIG. 11, the other arrangement example 3 of the external connecting terminals is explained. As shown in FIG. 11, a biological information detection apparatus 10g includes a first convex portion 35g having end portions 51g, 37g on both sides and a second convex portion 36g having end portions 52g, 38g on both sides like those of the first embodiment. When the back surface 33r of the bottom case (lid part) 33 is seen from the Z-axis direction, the first convex portion 35g and the second convex portion 36g are extended along the circumference of the detection part 92, protrude from the back surface 33r of the bottom case 33 in the same direction as the protrusion direction of the detection part 92, and are provided in the positions with the detection part 92 in between in the X-axis direction.

Further, first external connecting terminals 45g exposed on the contact surface side with the test subject SK are provided in the first convex portion 35g. Further, second external connecting terminals 46g exposed on the contact surface side with the test subject SK are provided in the second convex portion 36g like the first convex portion 35g. The first external connecting terminals 45g and the second external connecting terminals 46g are connecting terminals for connecting terminals for signal transmission between an external apparatus and themselves and power supply.

As described above, the first external connecting terminals 45g and the second external connecting terminals 46g are arranged, and thereby, damage on the first external connecting terminals 45 and the second external connecting terminals 46 from outside may be suppressed. Further, the first external connecting terminals 45g and the second external connecting terminals 46g are provided on the top parts of the protruding first convex portion 35g and second convex portion 36g, and thereby, connection may be easily made. Furthermore, the space efficiency according to the arrangement of the first external connecting terminals 45g and the second external connecting terminals 46g may be improved.

Note that the first external connecting terminals 45g and the second external connecting terminals 46g may be provided on surfaces recessed toward the case side from the surfaces on the contact surface side with the test subject SK of the respective first convex portion 35g and second convex portion 36g.

Further, the first external connecting terminals 45g and the second external connecting terminals 46g provided in the first convex portion 35g and the second convex portion 36g, respectively, are not to limited to the configurations exposed on the contact surface side with the test subject SK. The first external connecting terminals 45g and the second external connecting terminals 46g may be provided to be exposed on e.g. outer surfaces (outer wall surfaces) or inner surfaces (inner wall surfaces) of the first convex portion 35g and the second convex portion 36g.

The first external connecting terminals 45 and the second external connecting terminals 46 shown in FIG. 3 may be provided in a region on the opposite side to the detection part 92 side via the first convex portion 35 and the second convex portion 36 in the plan view in which the back surface 33r is seen from the Z-axis direction. As described above, the external connecting terminals (first external connecting terminals 45 and second external connecting terminals 46) are located on the outer side than the first convex portion 35 and the second convex portion 36, and thereby, the connection to the external connecting terminals (first external connecting terminals 45 and second external connecting terminals 46) may be easily made.

### Other Configuration Examples of First Convex Portion (Second Convex Portion)

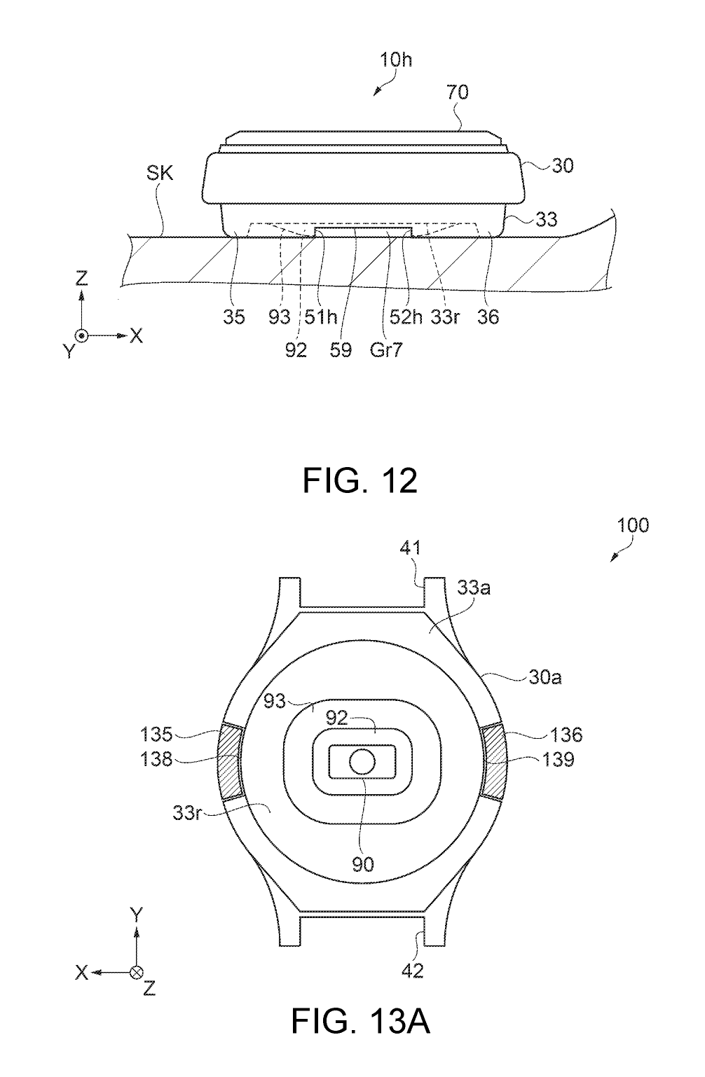

Next, referring to FIG. 12, another arrangement example of the first convex portion (second convex portion) will be explained. FIG. 12 is a side view showing the other configuration example of the first convex portion (second convex portion) as seen from the same direction as that in FIG. 4A. In the configurations of the first convex portion (second convex portion) in the above described embodiments and other arrangement examples, the bottom part of the groove portion dividing the first convex portion (second convex portion) is not necessarily the back surface 33r of the bottom case 33.

The specific example is made. As shown in FIG. 12, a biological information detection apparatus 10h of the configuration example has a first convex portion 35 and a second convex portion 36 like those of the above described first embodiment. Further, the biological information detection apparatus 10h has a groove portion located between one end 51h of the first convex portion 35 and one end 52h of the second convex portion 36, e.g., a seventh groove portion Gr7. The seventh groove portion Gr7 has a bottom part 59 not reaching the back surface 33r of the bottom case 33. In other words, the bottom part 59 of the seventh groove portion Gr7 is located between the contact surfaces of the first convex portion 35 and the second convex portion 36 on the test subject SK side and the back surface 33r of the bottom case 33. The same effects as those described above may be exerted in the configuration having the groove portion like the seventh groove portion Gr7.

### Second Embodiment

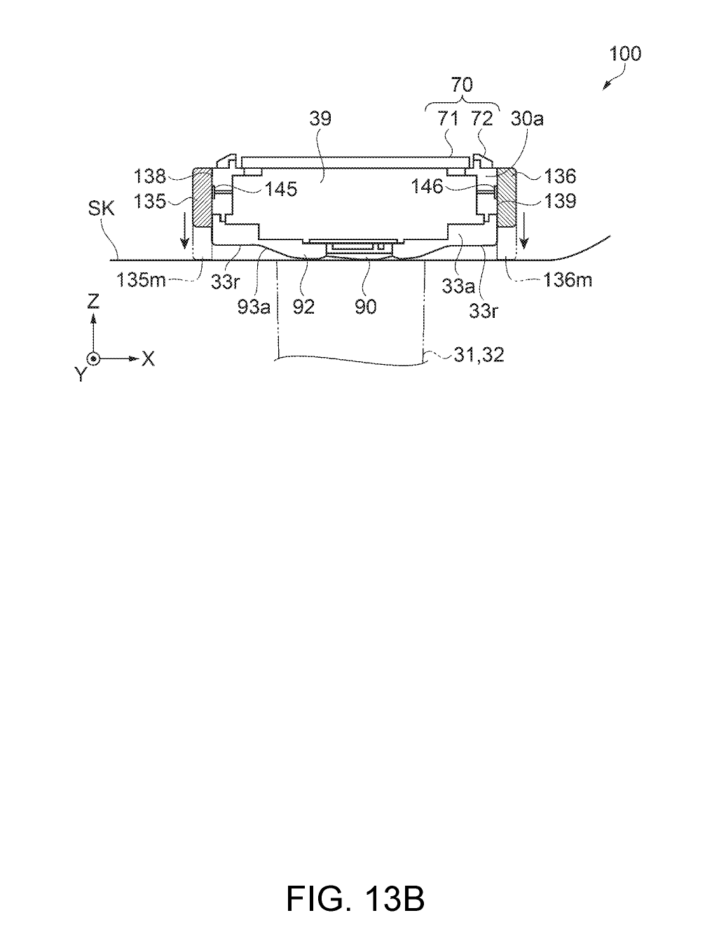

A configuration example of a biological information detection apparatus according to the second embodiment of the invention will be explained using FIGS. 13A and 13B. FIG. 13A shows the configuration example of the biological information detection apparatus according to the second embodiment in a plan view of a case as seen from the back surface side. FIG. 13B shows the biological information detection apparatus according to the second embodiment in a side view of the case as seen from an extension direction (connected direction) of band parts. Note that, as below, the explanation will be made with the display surface side of the biological information detection apparatus as the top side or front side and the opposite side to the display surface (the side in contact with the test subject when worn) as the bottom side or back side. Further, in FIGS. 13A and 13B, for convenience of explanation as is the case of the first embodiment, the explanation will be made using three axes orthogonal to one another as an X-axis, Y-axis, Z-axis, and an axis perpendicular to the surface of a glass plate 71 is the Z-axis and the direction in which a pair of band parts (first band part 31 and second band part 32) placed with the case in between are arranged is the Y-axis. Furthermore, as below, “axis direction” refers to “direction along axis”.

The biological information detection apparatus 100 according to the second embodiment is attached to a test subject SK as shown in FIG. 13B like that in the above described first embodiment. As is the case of the first embodiment, in the biological information detection apparatus 100, a bottom case (lid part) 33a as a case in which a sensor part 90 containing a light sensor that detects biological information of the test subject SK is provided and a top case 30a located on the opposite side to the attachment side to a user are provided. Further, band parts (first band part 31 and second band part 32) are respectively connected to both sides of a case part including the bottom case 33a and the top case 30a along the Y-direction. As below, the biological information detection apparatus 100 according to the second embodiment will be explained in detail, however, the same configurations as those of the above described first embodiment have the same signs and their explanation may be omitted.

As shown in FIGS. 13A and 13B, the biological information detection apparatus 100 according to the second embodiment has a first convex portion 135 and a second convex portion 136 provided on both sides along the outer circumference of the case part in a direction (X-axis direction) crossing the direction (Y-axis direction) in which the first band part 31 and the second band part 32 provided with the case part including the bottom case 33a and the top case 30a in between are arranged. Note that the first convex portion 135 and the second convex portion 136 are provided to be movable with respect to the case part (top case 30a).

In the top case 30a, storage portions 138, 139 recessed from the outer edges toward inside are provided on both sides in the X-axis direction. When not in use, the first convex portion 135 and the second convex portion 136 are housed in the storage portions 138, 139. When the first convex portion 135 and the second convex portion 136 are used, the first convex portion 135 and the second convex portion 136 move toward the back side with respect to the top case 30a (in directions of arrows shown in FIG. 13B), and fixed by fixing portions (not shown) in positions projecting from the top case 30a. In FIG. 13B, the fixing portions are shown as a first convex portion 135m and a second convex portion 136m by two-dashed dotted lines. In this manner, the first convex portion 135 and the second convex portion 136 function as spacers for adjusting gaps produced between the back surface 33r of the bottom case 33a and the test subject SK.

It is preferable that the first convex portion 135 and the second convex portion 136 are formed using a soft material including e.g. urethane resin, silicone resin, elastomer resin, fluorine resin, polystyrene resin, and polyethylene resin. Or, it is preferable to provide the above described soft material in the tip end portions including the contact surfaces of the first convex portion 135 and the second convex portion 136 with the test subject SK. As described above, the soft material is used for the first convex portion 135 and the second convex portion 136, and thereby, the uncomfortable feeling of the user may be reduced.

Note that, as shown in FIG. 13B, external connecting terminals (first external connecting terminals 145 and second external connecting terminals 146) may be provided in the top case 30a to be exposed to the storage portions 138, 139. In this case, the first convex portion 135 and the second convex portion 136 housed in the storage portions 138, 139 function as cover members of the external connecting terminals (first external connecting terminals 145 and second external connecting terminals 146), and thereby, may suppress stain and breakage of the external connecting terminals (first external connecting terminals 145 and second external connecting terminals 146).

According to the above described biological information detection apparatus 100 according to the second embodiment, the first convex portion 135 and the second convex portion 136 may be moved as appropriate and placed in the gaps produced between the back surface 33r of the bottom case 33 and the test subject SK. In this manner, the first convex portion 135 and the second convex portion 136 may be easily placed (attached).

Further, the first convex portion 135 and the second convex portion 136 are housed in the storage portions 138, 139 (top case 30a), and thereby, the first convex portion 135 and the second convex portion 136 are not projected when not necessary and the designability may be improved.

### Third Embodiment

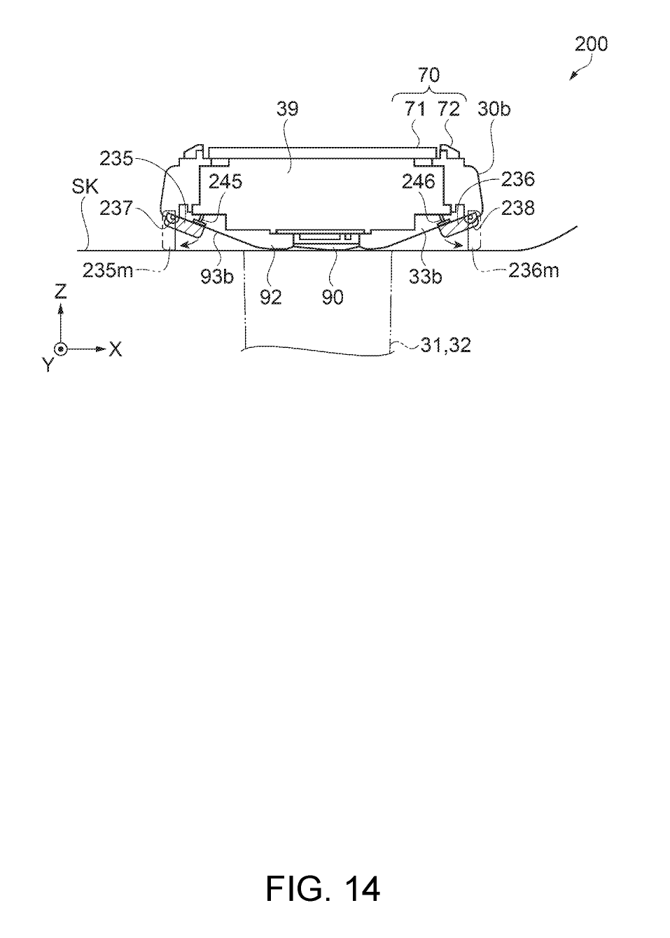

A configuration example of a biological information detection apparatus according to the third embodiment of the invention will be explained using FIG. 14. FIG. 14 shows the configuration example of the biological information detection apparatus according to the third embodiment in a side view of a case as seen from an extension direction (connected direction) of band parts. Note that, as below, the explanation will be made with the display surface side of the biological information detection apparatus as the top side or front side and the opposite side to the display surface (the side in contact with the test subject when worn) as the bottom side or back side. Further, in FIG. 14, for convenience of explanation, as is the case of the first embodiment, the explanation will be made using three axes orthogonal to one another as an X-axis, Y-axis, Z-axis, and an axis perpendicular to the surface of a glass plate 71 is the Z-axis and the direction in which a pair of band parts (first band part 31 and second band part 32) placed with the case in between are arranged is the Y-axis. Furthermore, as below, “axis direction” refers to “direction along axis”.

The biological information detection apparatus 200 according to the third embodiment is attached to a test subject SK like that in the above described first embodiment. As is the case of the first embodiment, in the biological information detection apparatus 200, a bottom case (lid part) 33b as a case in which a sensor part 90 containing a light sensor that detects biological information of the test subject SK is provided and a top case 30b located on the opposite side to the attachment side to a user are provided. Further, band parts (first band part 31 and second band part 32) are respectively connected to both sides of a case part including the bottom case 33b and the top case 30b along the Y-direction. As below, the biological information detection apparatus 200 according to the third embodiment will be explained in detail, however, the same configurations as those of the above described first embodiment have the same signs and their explanation will be omitted.

As shown in FIG. 14, the biological information detection apparatus 200 according to the third embodiment has a first convex portion 235 and a second convex portion 236 provided on both sides along the outer circumference of the case part in a direction (X-axis direction) crossing the direction (Y-axis direction) in which the first band part 31 and the second band part 32 provided with the case including the bottom case 33b and the top case 30b in between are arranged. The first convex portion 235 and the second convex portion 236 are connected to be rotationally moved to the case part (top case 30b). In the detailed explanation, the first convex portion 235 is rotatable about a rotation shaft 237 fixed to one outer edge (in the −X-axis direction) of the top case 30b and moves the position thereof by the rotation. Further, the second convex portion 236 is rotatable about a rotation shaft 238 fixed to the other outer edge (in the +X-axis direction) of the top case 30b and moves the position thereof by the rotation.

When the first convex portion 235 and the second convex portion 236 are not used as spacers, the first convex portion 235 and the second convex portion 236 are fixed by fixing portions (not shown) along a slope 93b on the back side of the bottom case 33b. When the first convex portion 235 and the second convex portion 236 are used, the first convex portion 235 and the second convex portion 236 are rotated and moved in directions of arrows in the drawing about the rotation shaft 237 fixed to the top case 30b, and the first convex portion 235 and the second convex portion 236 are projected from the top case 30b and held. In FIG. 14, the positions in which the portions are held are shown as a first convex portion 235m and a second convex portion 236m by two-dashed dotted lines. In this manner, the first convex portion 235 and the second convex portion 236 function as spacers for adjusting gaps produced between the slope 93b of the bottom case 33b and the test subject SK.

It is preferable that the first convex portion 235 and the second convex portion 236 are formed using a soft material including e.g. urethane resin, silicone resin, elastomer resin, fluorine resin, polystyrene resin, and polyethylene resin. Or, it is preferable to provide the above described soft material in the tip end portions including the contact surfaces of the first convex portion 235 and the second convex portion 236 with the test subject SK. As described above, the soft material is used for the first convex portion 235 and the second convex portion 236, and thereby, the uncomfortable feeling of the user may be reduced.

Note that external connecting terminals (first external connecting terminals 245 and second external connecting terminals 246) may be provided in the bottom case 33b to be exposed to the slope 93b with respect to the fixed positions when the first convex portion 235 and the second convex portion 236 are not used as spacers. In this case, the first convex portion 235 and the second convex portion 236 fixed to the slope 93b when not in use as spacers function as cover members of the external connecting terminals (first external connecting terminals 245 and second external connecting terminals 246), and thereby, may suppress stain and breakage of the external connecting terminals (first external connecting terminals 245 and second external connecting terminals 246).

According to the above described biological information detection apparatus 200 according to the third embodiment, the first convex portion 235 and the second convex portion 236 may be moved from the fixing portions (not shown) in the rotation directions as appropriate. Thereby, the portions may be placed in the gaps produced between the slope 93b of the bottom case 33b and the test subject SK located outside of the detection part 92. In this manner, the first convex portion 235 and the second convex portion 236 may be easily placed (attached).

Further, the first convex portion 235 and the second convex portion 236 are fixed along the slope 93b, and thereby, the first convex portion 235 and the second convex portion 236 are not projected when the first convex portion 235 and the second convex portion 236 are not necessary and the designability may be improved.

### Fourth Embodiment

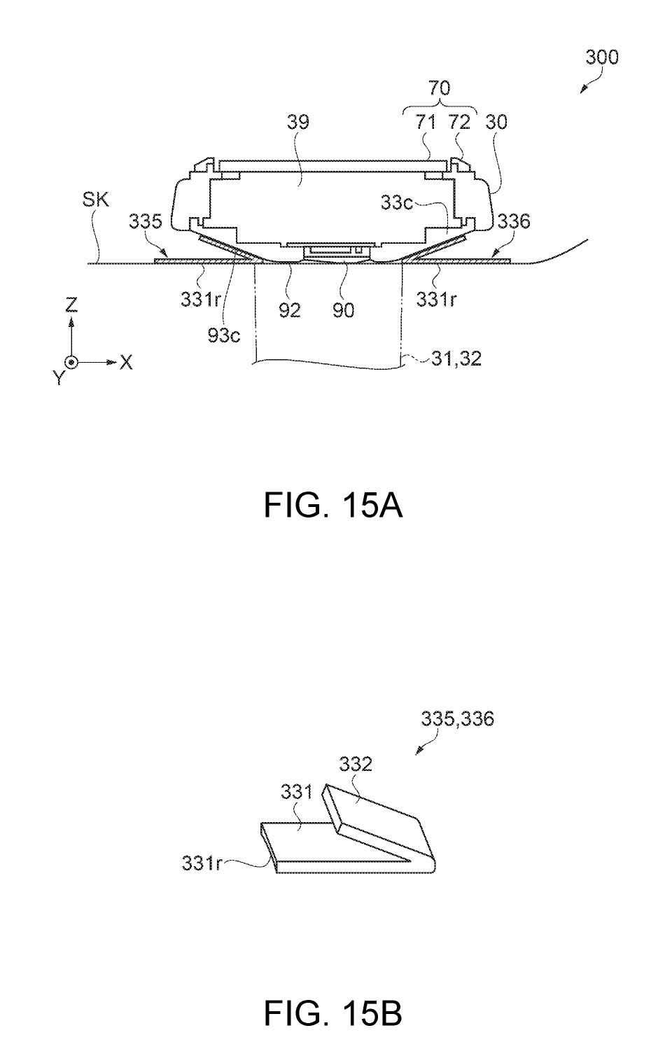

A configuration example of a biological information detection apparatus according to the fourth embodiment of the invention will be explained using FIGS. 15A and 15B. FIG. 15A shows the configuration example of the biological information detection apparatus according to the fourth embodiment in a side view of a case as seen from an extension direction (connected direction) of band parts. FIG. 15B is a perspective view showing a first convex portion (second convex portion) in the fourth embodiment. Note that, as below, the explanation will be made with the display surface side of the biological information detection apparatus as the top side or front side and the opposite side to the display surface (the side in contact with the test subject when worn) as the bottom side or back side. Further, in FIG. 15A, for convenience of explanation, as is the case of the first embodiment, the explanation will be made using three axes orthogonal to one another as an X-axis, Y-axis, Z-axis, and an axis perpendicular to the surface of a glass plate 71 is the Z-axis and the direction in which a pair of band parts (first band part 31 and second band part 32) placed with the case in between are arranged is the Y-axis. Furthermore, as below, “axis direction” refers to “direction along axis”.

The biological information detection apparatus 300 according to the fourth embodiment is attached to a test subject SK as shown in FIG. 15A like that in the above described first embodiment. As is the case of the first embodiment, in the biological information detection apparatus 300, a bottom case (lid part) 33c as a case in which a sensor part 90 containing a light sensor that detects biological information of the test subject SK is provided and a top case 30 located on the opposite side to the attachment side to a user are provided. Further, band parts (first band part 31 and second band part 32) are respectively connected to both sides of a case part including the bottom case 33c and the top case 30 along the Y-direction. As below, the biological information detection apparatus 300 according to the fourth embodiment will be explained in detail, however, the same configurations as those of the above described first embodiment have the same signs and their explanation will be omitted.

As shown in FIG. 15A, the biological information detection apparatus 300 according to the fourth embodiment has a spacer 335 as a first convex portion and a spacer 336 as a second convex portion provided on both sides of the outer circumference of the case part in a direction (X-axis direction) crossing the direction (Y-axis direction) in which the first band part 31 and the second band part 32 provided with the case including the bottom case 33c and the top case 30 in between are arranged. Note that the spacers 335, 336 are arbitrarily detachable from the bottom case 33c.

The spacers 335, 336 may be attached as appropriate when the biological information detection apparatus 300 is attached to the test subject SK. The spacers 335, 336 are inserted into gaps produced between a slope 93c of the back side of the bottom case 33c and the test subject SK from both sides along the X-axis direction and function as spacers when the biological information detection apparatus 300 is attached to the test subject SK.

As shown in FIG. 15B, the spacers 335, 336 each includes a bottom portion 331 on the side of the test subject SK and a tilted portion 332 folded at an acute angle along the slope 93c (see FIG. 15A) from the bottom portion 331. Note that it is preferable that a back surface 331r of the bottom portion 331 as a surface on the side in contact with the test subject SK has e.g. a curved shape along the shape of the wrist of the user.

Further, it is preferable that the spacers 335, 336 are formed using a soft material including e.g. urethane resin, silicone resin, elastomer resin, fluorine resin, polystyrene resin, and polyethylene resin. Or, it is preferable to provide the above described soft material in parts of the back surface 331r as the contact surfaces with the test subject SK. As described above, the soft material is used for the spacers 335, 336, and thereby, the uncomfortable feeling of the user may be reduced.

According to the above described biological information detection apparatus 300 according to the fourth embodiment, the spacer 335 as the first convex portion and the spacer 336 as the second convex portion may be used only when necessary, and the designability may be improved.

### Modified Example of Biological Information Detection Apparatus

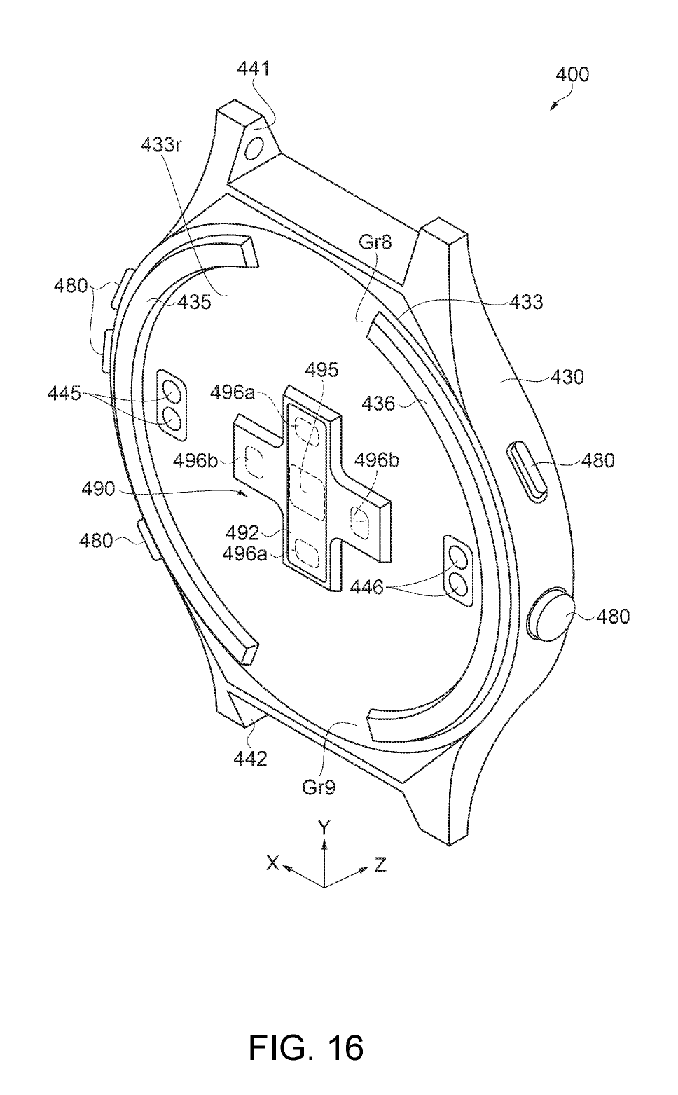

A modified example according to the biological information detection apparatus according to the invention will be explained using FIG. 16. FIG. 16 is a bottom (back surface) perspective view showing a biological information detection apparatus according to the modified example. As below, the explanation will be made with the display surface side of the biological information detection apparatus as the top side or front side and the opposite side to the display surface (the side in contact with the test subject when worn) as the bottom side or back side. Further, in FIG. 16, for convenience of explanation, as is the case of the first embodiment, the explanation will be made using three axes orthogonal to one another as an X-axis, Y-axis, Z-axis, and an axis perpendicular to the surface of a glass plate 71 is the Z-axis and the direction in which a pair of band parts placed with the case in between are arranged is the Y-axis. Furthermore, as below, “axis direction” refers to “direction along axis”.

The biological information detection apparatus 400 according to the modified example is attached to a test subject SK (not shown in FIG. 16) like that in the above described first embodiment. As is the case of the first embodiment, in the biological information detection apparatus 400, a bottom case (lid part) 433 as a case in which a sensor part 490 containing a light sensor that detects biological information of the test subject SK is provided and a top case 430 located on the opposite side to the attachment side to a user are provided. Further, band parts (not shown) are respectively connected to both sides of a case part including the bottom case 433 and the top case 430 along the Y-direction.

The configuration of the biological information detection apparatus 400 is explained in further detail. The biological information detection apparatus 400 includes the case part including the bottom case 433 and the top case 430, operation parts 480, and the sensor part 490. Note that the biological information detection apparatus 400 of the modified example has the configurations relating to the first band part 31, the second band part 32, the fixing part (free loop, fixed loop) 40, and the connecting part (buckle) 50 and the configurations relating to the display part 70 explained in the first embodiment, however, the parts are the same as those of the first embodiment and their illustration is omitted and their explanation is omitted.

The bottom case 433 and the top case 430 forming the case part may be formed using e.g. a metal such as stainless steel or resin. Note that the case part is not necessarily separated into the bottom case 433 and the top case 430, but a configuration in which a case having an integrated structure (corresponding to the top case 430) and a back cover (corresponding to the bottom case 433) are provided on the attachment side of the case part to the user may be employed.

On the case outer surface of the bottom case 433 located on the test subject SK side, a detection part 492 housing the sensor part 490 containing the light sensor that detects biological information of the test subject SK and a back surface 433r placed on the outer side than the outer edge of the detection part 492 (the side on which the outer edge of the bottom case 433 is located) are provided. Further, a pair of external connecting terminals (first external connecting terminals 445 and second external connecting terminals 446) are provided on the back surface 433r.

On the side surface of the biological information detection apparatus 400, e.g. the operation parts 480 including a plurality of buttons that enable switching of detection modes by the sensor part 490 and switching of display modes displayed on the display part (not shown) are provided. The operation parts 480 may receive operation by the user.

In an internal space formed by the bottom case 433, the top case 430, a glass plate (not shown), etc., the display part including a dial plate (not shown), a control part (not shown) that processes data detected by the sensor part 490 and controls display contents of the display part, a battery (not shown), etc. are housed.

The sensor part 490 is housed in the detection part 492 provided to protrude in a convex shape from the back surface 433r as the case outer surface of the bottom case 433 toward the test subject SK side. In other words, the sensor part 490 further protrudes toward the test subject SK side (Z-axis direction) than the back surface 433r. The detection part 492 of the modified example has an outer shape of the so-called cross shape in which rectangular portions extending in the X-axis direction and the Y-axis direction cross. Note that the outer shape of the detection part 492 is not limited to the cross shape, but may be any shape. The detection part 492 houses the sensor part 490 containing the light sensor in the center part. The sensor part 490 is pressed against the test subject SK with the detection part 492 to detect biological information of the test subject SK.

In the sensor parts 490, two light emitting portions 496a and a light receiving portion 495 placed between the two light emitting portions 496a are provided. The light emitting portions 496a output light to the test subject SK. The light receiving portion 495 receives light (reflected light, transmitted light) from the test subject SK. For example, when the light emitting portions 496a output light to the test subject SK and the light is reflected by the test subject SK (blood vessel), the light receiving portion 495 receives the reflected light and may detect the biological information. The light receiving portion 495 may be realized by e.g. a light receiving element such as a photodiode and the light emitting portions 496a may be realized by light emitting elements e.g. LEDs. Note that, in the above description, the arrangement example in which the two light emitting portions 496a are along the Y-axis is shown, however, two light emitting portions 496b may be arranged along the X-axis as shown by imaginary lines (dashed-dotted lines) in FIG. 16. Also, in this case, the light receiving portion 495 is placed between the two light emitting portions 496b.

A pulsimeter is taken as an example. The light from the light emitting portions travels within the test subject, and is diffused or scattered by epidermis, dermis, subcutaneous tissue etc. Then, the light reaches the blood vessel (part to be detected) and is reflected. In this regard, part of the light is absorbed by the blood vessel. Then, absorption of the light in the blood vessel changes due to the pulse rate and the light quantity of the reflected light also changes, and thereby, the pulse rate etc. as biological information may be detected by the light receiving portion receiving the reflected light and detecting the change of the light quantity.

Further, in the plan view of the back surface 433r of the bottom case 433 from the Z-axis direction, a first convex portion 435 and a second convex portion 436 placed in positions with the detection part 492 in between in the X-axis direction are provided on the back surface 433r in a pair. In other words, the first convex portion 435 and the second convex portion 436 are provided in the positions with the detection part 492 in between in the X-axis direction as a direction crossing the direction in which band attachment portions 441, 442 are provided, i.e., the direction (Y-axis direction) in which the band parts are connected. Further, the first convex portion 435 and the second convex portion 436 protrude from the back surface 433r of the bottom case 433 to surround the detection part 492 in the same direction as the protrusion direction of the detection part 492 (Z-axis direction). The heights of the first convex portion 435 and the second convex portion 436 from the back surface 433r may be generally the same as the height of the detection part 492 from the back surface 433r, and more preferably the same. Further, the first convex portion 435 and the second convex portion 436 are extended in an arc shape along at least a part of the outer edge of the bottom case 433. In the extension direction of the first convex portion 435 and the second convex portion 436, in regions overlapping with the Y-axis direction, two groove portions Gr8, Gr9 cut out from the detection part 492 side over the outer edge of the bottom case 433 are provided. Further, the first convex portion 435 and the second convex portion 436 are divided by the two groove portions Gr8, Gr9 provided one by one on both sides with the sensor part 490 in between in the Y-axis direction. In other words, there are two regions in which the first convex portion 435 and the second convex portion 436 are not joined.

As described above, regarding the first convex portion 435 and the second convex portion 436, the pair of first convex portion 435 and second convex portion 436 divided by the two groove portions Gr8, Gr9 cut out from the detection part 492 side over the outer edge side of the bottom case 433 are disposed, and thereby, when the biological information detection apparatus 400 is attached to the attachment part (test subject SK) of the user, the tilt of the case part (bottom case 433 and the top case 430) with respect to the directions on both sides with the detection part 492 in between may be reduced.

Note that the first convex portion 435 and the second convex portion 436 may be integrally provided with the bottom case 433 or separately molded from the bottom case 433 and attached to the back surface 433r of the bottom case 433 by bonding.

In the plan view of the back surface 433r of the bottom case 433 as seen from the Z-axis direction, the external connecting terminals (first external connecting terminals 445 and second external connecting terminals 446) are provided in a region between the detection part 492 and the first convex portion 435 and a region between the detection part 492 and the second convex portion 436 in the X-axis direction. Note that the first external connecting terminals 445 and the second external connecting terminals 446 are the same as those of the first embodiment and their explanation is omitted.

According to the above described biological information detection apparatus 400 according to the modified example, in the plan view of the back surface 433r of the bottom case 433 as seen from the Z-axis direction, the first convex portion 435 and the second convex portion 436 are provided in the pair in the positions with the detection part 492 in between in the X-axis direction. Thereby, when the apparatus main body formed by the top case 430, bottom case 433, etc. is attached to the attachment part (test subject SK) of the user, in addition to the detection part 492, the first convex portion 435 and the second convex portion 436 come into contact with the test subject SK, and the tilt of the case part about the detection part 492 as a fulcrum may be reduced. In other words, even when the wearer does heavy exercise and the test subject SK moves, the state in which the sensor part 490 (detection part 492) is pressed against the test subject SK with given pressure can be maintained. As described above, the attachment (fixation) attitude of the biological information detection apparatus 400 is stable, and thereby, false detection of the sensor signal according to the biological information including e.g. pulse wave and pulse rate may be prevented and the detection of the biological information may be stabilized.

Note that the first convex portion 435 and the second convex portion 436 are provided in the positions with the detection part 492 in between along the X-axis direction as the direction crossing the direction in which the band parts are connected (Y-axis direction). In the X-axis direction, swing and tilt are relatively easy to occur, however, the first convex portion 435 and the second convex portion 436 are provided in the X-axis direction, and thereby, occurrence of swing and tilt of the biological information detection apparatus 400 may be made harder.

Further, by the groove portions Gr8, Gr9 provided between the first convex portion 435 and the second convex portion 436, the region in which the detection part 492 is located is not an enclosed space, but communicates with regions outside of the first convex portion 435 and the second convex portion 436, and retention of moisture (water) due to e.g. sweat in the region in which the detection part 492 is located may be reduced. Thereby, unstable detection due to moisture (water) may be reduced and the detection of the biological information by the sensor part 490 may be stabilized.

In addition, the first convex portion 435 and the second convex portion 436 are provided to surround the outer circumference of the sensor part 490 and function as light-blocking walls, and outside light including disturbance light entering the sensor part 90 can be blocked or suppressed.

What is claimed is:

1. A biological information detection apparatus comprising:
a case;
a detector that houses a light receiving sensor configured to receive light from a test subject, the detector protruding from an outer surface of the case on the test subject side; and
a convex part with a cutout provided in at least a portion of the convex part in a plan view and protruding from the test subject side of the case.

2. The biological information detection apparatus according to claim 1, wherein the convex part includes a first convex portion and a second convex portion, and
the cutout is disposed in a region in which the first convex portion and the second convex portion are not joined in the plan view.

3. The biological information detection apparatus according to claim 2, wherein the first convex portion and the second convex portion are provided closer to an outer edge side of the case than the detector.

4. The biological information detection apparatus according to claim 2, wherein a surface of the region in which the first and second convex portions are provided is the same surface as the case outer surface.

5. The biological information detection apparatus according to claim 2, further comprising a pair of bands provided with the case in between in the plan view,
wherein the first convex portion and the second convex portion are provided along a direction crossing a direction in which the pair of bands are arranged in the plan view.

6. The biological information detection apparatus according to claim 2, wherein external connecting terminals are provided in a region between the detector and the first convex portion and a region between the detector and the second convex portion.

7. The biological information detection apparatus according to claim 2, wherein external connecting terminals are located in a region opposite to the detector side with respect to the first convex portion and the second convex portion in the plan view.

8. The biological information detection apparatus according to claim 2, wherein external connecting terminals are located on a surface of the region.

9. The biological information detection apparatus according to claim 2, wherein external connecting terminals are located in at least one of the first convex portion and the second convex portion.

10. The biological information detection apparatus according to claim 2, wherein the first convex portion and the second convex portion are detachable with respect to the case.

11. The biological information detection apparatus according to claim 2, wherein the first convex portion and the second convex portion are provided movably with respect to the case.

12. The biological information detection apparatus according to claim 2, wherein the first convex portion and the second convex portion are connected to be rotationally moved with respect to the case.

13. The biological information detection apparatus according to claim 2, wherein the case includes storage portions that house the first convex portion and the second convex portion.

14. The biological information detection apparatus according to claim 1, wherein a section of a portion of the convex part in contact with the test subject has a curved shape.

15. The biological information detection apparatus according to claim 1, wherein the convex portion is circumferentially provided to surround the detector in the plan view.

16. A biological information detection wristwatch comprising:
a case having a front surface and a back surface;
a detector that includes a light receiving sensor configured to receive light from a test subject, the detector protruding from a central portion of the back surface of the case; and
a plurality of convex protrusions provided on the back surface of the case configured to surround the detector, the convex portions each extending in an arc shape.
